(19) United States
(12) Patent Application Publication
(10) Pub. No.: US 2003/0009138 A1
(43) Pub. Date: Jan. 9, 2003

(54) Aromatherapy footwear

(76) Inventors: Cindy N. Freeman, Calabasas, CA (US); Catherine L. Nunes, Los Altos, CA (US)

Correspondence Address: Mandel & Adriano, Suite 710, 55 S. Lake Avenue, Pasadena, CA, 91101, US

(21) Appl. No.: 10/163,241

(22) Filed: Jun. 4, 2002

Related U.S. Application Data
(60) Provisional application No. 60/295,730, filed on Jun. 4, 2001.

(51) Int. Cl. A61M 35/00; A61B 19/00

(52) U.S. Cl. 604/290; 604/289; 128/898

(57) ABSTRACT

The present invention is footwear and footwear components including insole pads, inserts and liners containing plant-derived essential oils and/or dried plant products applied to or constructed within the footwear and footwear components to deliver the comfort, disinfectant and/or therapeutic benefits of aromatherapy through direct contact with the wearer's feet with the volatile components of the plant derived oils and/or plant products.

This application claims priority to provisional application, U.S. Serial No. 60/295,730, filed Jun. 4, 2001, the contents of which are hereby incorporated by reference in their entirety into this application.

### FIELD OF THE INVENTION

This invention relates to aromatherapy, and in particular to the application of essential oils or volatile plant material to human feet through footwear and/or footwear components, to increase comfort and provide therapeutic benefits.

### BACKGROUND OF THE INVENTION

In the footwear business, the focus has been on improving comfort and performance by changing the shape, fabric and design, including varying the materials and technology that provide padding and cushioning of the feet of the wearer. Most inventions have heretofore concerned developing processes of molding and contouring the innersoles and arch position of the footwear, evolving padding elements (such as air or gel), or increasing the air circulation within the shoe or footpad.

Separately, there has been a revitalized interest in developing products and services centered on the treatment of human feet. Reflexology, massage and foot spa treatments have received renewed interest, recognizing the importance of foot care for pain relief, increased comfort and other therapeutic value to a person's well being. Additionally, there has been proliferation of foot-specific lotions, balms, salt scrubs and personal care products many of which have incorporated the therapeutic value of essential oils and/or aromatherapy plant byproducts. One of the most receptive places for applying essential oils to a person is the feet. Reflexology concerns identifying regions of the human feet that are believed to affect organs and other areas of the body.

Essential oils are highly concentrated, volatile and non-greasy liquids that are distilled from the flowers, leaves, stems, roots, bark, grass and seeds of botanicals. Although many oils have become known for their fragrance properties, they also have been known for their therapeutic benefits. These oils are complex in their molecular constructs and contain constituents like phenols, terpenes and ketones. Many essential oils are used as active ingredients in prescription drugs or were the inspiration for chemical copying. For example, Colperin^{R}, the tradename for a prescription drug, is essential oil of peppermint used in the treatment of arthritis and rheumatism.

Another aspect of essential oils has been their ability to be absorbed into the blood stream through the trans-dermal application on skin. For example, garlic, even when applied to the skin, can be later detected in the breath.

Certain essential oils have been known to inhibit bacterial growth, thereby preventing and destroying odors and fighting bacterial growth. Studies have shown that environments where essential oils and oils of cinnamon and oregano have been introduced have stopped and eradicated the growth of bacteria.

Previously unknown for footwear, or footwear components such as insole pads or liners, has been the incorporation of the comfort and therapeutic benefits of essential oils and plant-derived products, constructed or applied into the footwear so as to be directed to contact a person's feet for therapeutic benefits.

Accordingly, it is an object of the invention to allow the volatile elements of essential oils and/or plant products to come in contact with a wearer's feet for therapeutic benefits through the use of footwear incorporating the essential oils and/or plant products.

It is a further object of this invention to provide components of footwear in which insole footpads, liners or inserts are supplied or constructed with essential oils and/or plant products.

Another object of the invention is to provide a multilayered component of footwear, in which an inner layer is supplied or constructed with essential oils and/or plant products in contact with an outer layer of natural and/or synthetic porous fabric so as to draw and distribute the volatile elements of the oils and/or plant products through the footwear, to contact the feet of the wearer.

It is also an object of this invention to direct essential oils and/or plant products in footwear into contact with specific areas of the wearer's feet for therapeutic benefits.

It is a further object of this invention to select particular essential oils, oil blends and/or plant-derived materials and blends for use in footwear, to provide benefits to footwear users, including, reducing the dryness of skin, eliminating bacterial growth and odor; and soothing tender areas and sore muscles.

### SUMMARY OF THE INVENTION

The objects of this invention are accomplished by providing footwear and/or footwear components, to which essential oils and/or plant products may be applied, or the oils and plant products may be constructed within the footwear and/or components, to provide aromatherapy benefits to the wearer's feet. The invention provides methods for applying the oils and plant products directly to footwear, or to components of footwear, that are inserted during manufacture or afterward.

In specific embodiments, the objects of this invention are accomplished by methods of incorporating essential botanical oils and/or plant products directly onto the material of the footwear, or on or within footwear components such as insole footpads, liners or inserts, to deliver the benefits of aromatherapy to the wearer. The oils and/or plant materials may be provided within a multilayered insole footpad, liner or insert, for example on absorbent material within an outer layer of natural or synthetic porous material.

### BRIEF DESCRIPTION OF THE DRAWINGS

FIGS. 1A-1C is illustrations of three different embodiments of the application of essential oils and/or plant products onto footwear or footwear components, (FIG. 1A is an insole footpad having an absorbent fabric outer covering to which the essential oil and/or plant products are directly applied; FIG. 1B is a multilayered insole footpad constructed of an inner absorbent layer infused with essential oils, or absorbent batting alternating with plant products, beneath a porous, natural or synthetic outer material; FIG. 1C is an insole footpad made of an absorbent natural plant product to which the essential oils and/or plant products are directly applied), as described in detail, infra.

FIGS. 2A-2C is illustrations depicting cross-sections of footwear components constructed according to the methods of the invention, (FIG. 2A is a cross-sectional view of an insole footpad showing an outer, natural or synthetic fabric cover to which the essential oils are topically applied, the inner material of the footpad, and a bottom leak-proof liner; FIG. 2B is a cross-sectional view of an insole footpad showing an outer, porous, natural or synthetic cover, an inner layer infused with essential oil and/or plant products, and a bottom leak-proof liner; FIG. 2C is a cross-sectional view of an insole footpad showing an outer, porous, natural or synthetic cover, an inner layer consisting of batted material mixed with plant products, and a bottom liner) as described in detail, infra.

FIGS. 3A-3B is illustrations of the assembled footwear of the invention (FIG. 3A is a slipper-like item of footwear with a insole footpad having an outer porous layer; FIG. 3B depicts two shapes of a removable liner footwear component having an outer porous layer on top of an inner, absorbent layer infused with essential oils and/or plant products), as described in detail, infra.

### DETAILED DESCRIPTION OF THE INVENTION

The present invention provides for application of essential oils or volatile plant materials directly onto or within footwear, including components of footwear such as insole footpads, liners or inserts, to increase comfort and provide therapeutic benefits to the wearer.

Depending on the selected essential oils or plant-derived products, varying effects and benefits are obtained by the wearer of the footwear incorporating these materials. The essential oils and/or plant products are selected to provide benefits that include, but are not limited to providing a pleasant fragrance, reducing the dryness of the wearer's skin on the feet, eliminating bacterial growth and odor in the footwear, and soothing tender areas or sore muscles of the feet.

A variety of known essential oils from botanicals exist and are readily obtainable. These oils may be used separately or blended to obtain optimum benefits. Examples of essential oils or plant products, include, but are not limited to, lavender, clary sage, mint, rosemary, orange, lemon, grapefruit, tangerine, sandalwood, eucalyptus. Other useful essential oils include tea tree oil and clove oil, which have long been recognized for their antiseptic benefits and incorporated into bath and body products.

In addition, combinations of essential oils and plant products may be used. Such combinations or “blends” include, but are not limited to, lavender and clary sage where the oil of clary sage is known to be a powerful relaxant and lavender is known for its calming effects and also reduces muscular pain from physical effort. The combination of lavender and clary sage can provide a skin soothing and calming effect. Mint and rosemary oils or plant material may be combined for refreshing skin, energizing and increasing circulation in the feet. In ancient Athens the men chose infused mint oil to massage on their arms for its clean and energizing effect. Stimulating mint offers extra benefits including boosting circulation, improving energy and a resistance to fatigue. Sandalwood and eucalyptus can be combined for deodorizing, cleansing and refreshing. Eucalyptus oil is stimulating and cleansing, as well as possessing both antiseptic and antiviral properties. Lemon, mandarin (tangerine) and grapefruit oils and/or plant materials can be combined to cleanse and purify, uplift and revive. Lemon and tangerine improves the skin by removing toxins. Lemon is mildly antiseptic and the essential oil is a stimulant, improving circulation.

Plant products can be used in fresh (“live”) or dried form in the invention. Dried plant materials containing essential oils, continue to hold these oils and may be readily obtained from plants known to have therapeutic benefits such as lavender buds. The oil is released by crushing the leaves. When dried plant materials are used in the footwear and/or footwear components of the invention, the foot of the wearer performs the mechanical crushing of the materials releasing the oils.

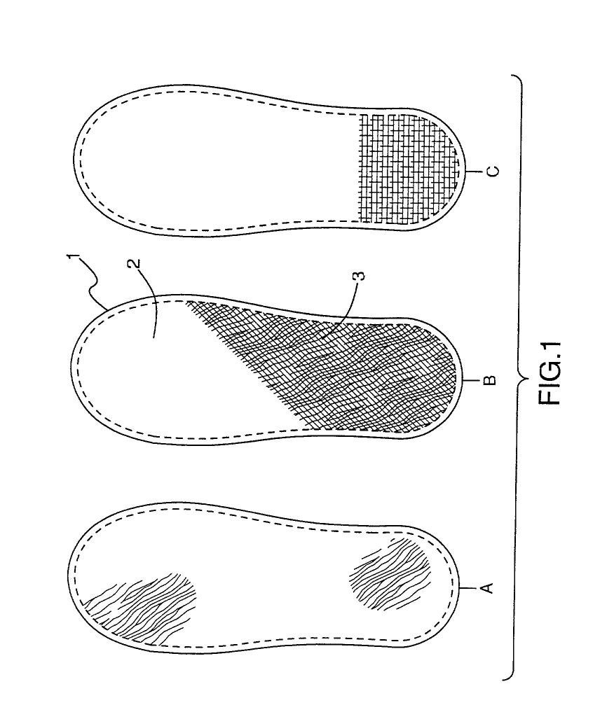

As shown in FIGS. 1A-C, the essential botanical oils or the plant products can be applied directly to materials used to manufacture the footwear or footwear components, for example by spraying, dipping or otherwise coating an absorbent fabric. Suitable materials include natural and synthetic absorbent materials such as cotton, or a naturally derived plant material, such as sea grass or loofah, that retains the oil and its volatile plant-derived elements, including phenols, terpenes and ketones. Alternatively, the oils or plant products can be applied to the finished footwear item, for example by spraying during or after manufacture.

Using the methods of the invention, the essential oils and/or plant products can also be applied or infused onto footwear components, such as an insole footpad, a liner or inserts made of absorbent material. As shown in FIG. 1B, a multilayered insole footpad component 1 may also be constructed, in which the essential oils and/or plant products are applied to an absorbent layer 2, such as a cotton fabric. The absorbent layer 2 is then placed underneath an outer fabric layer 3 that is porous and made of a natural material, such as sea grass or loofah, or a synthetic material, for example plastic mesh, which comes in contact with the feet of the wearer. This embodiment permits storing of the volatile elements of the oils or plant materials in the absorbent layer 2 for longer periods of time, and subsequent release of the volatile elements of the oils or plant materials through the porous outer layer 3. The multilayered component 1 provides a longer “shelf-life” for the footwear of the invention, and increases the amount of essential oils and/or plant materials that contact the wearer's feet.

To increase shelf-life, the essential oils may also be encapsulated using known encapsulation methods and applied to footwear materials to permit the release of the oils with wear over time.

To prevent leakage of the oils or plant materials, the bottom of the insole footpads, liners or inserts may be coated or lined with protective material such as plastic, rubber or other “leak-proof” substance. Alternatively, the footwear may be manufactured with a sole made of leak-proof substances.

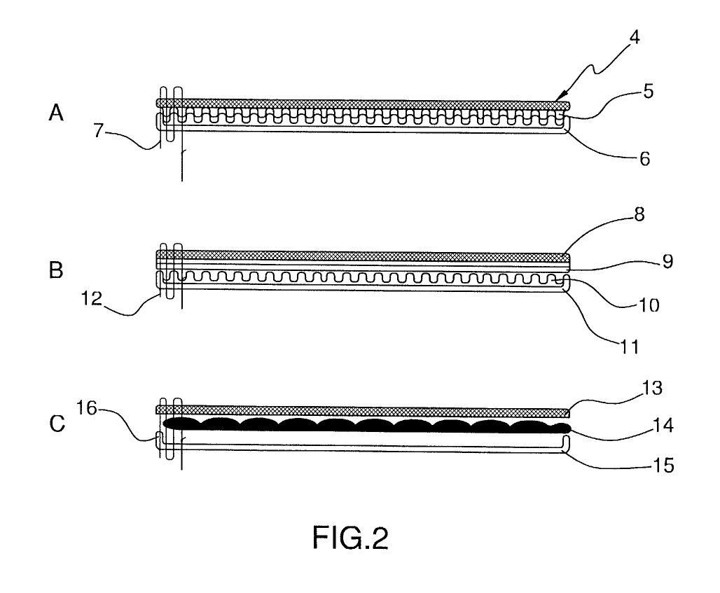

In another embodiment, multilayer footpads may be constructed, as shown in FIGS. 2A-2C. FIG. 2A shows a cross-section of a multilayer footpad for footwear consisting of an outer or top layer 4 of material to which essential oils are directly applied and an inner padding 5 with a bottom leak-proof liner 6. The footpad may be attached directly to the footwear, for example by stitching 7, or sealed to form a fully transferable, reusable footpad. FIG. 2B shows a multilayered footpad in which the outer cover 8 is a porous natural or synthetic material. The inner layer 9 is infused with essential oils and lies on top of padding 10 and a leak-proof bottom liner 11. The footpad may be attached directly to the footwear, for example by stitching 12, or sealed to form a fully transferable, reusable footpad. FIG. 2C depicts a multilayered footpad in which the outer layer 13 is a porous natural or synthetic material lying on top of batted pockets of cotton or other material interspersed with plant products 14 above a leak-proof liner 15. The footpad may be attached directly to the footwear, for example by stitching 16, or sealed to form a fully transferable, reusable footpad.

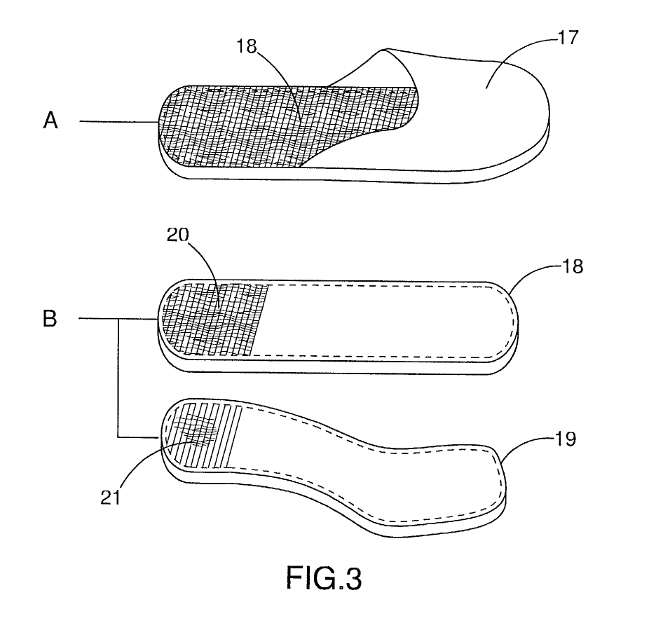

FIG. 3A shows an item of footwear, such as a slipper 17 having an insole footpad with a porous outer layer 18. FIG. 3B shows removable insole footpads 18 and 19 having a porous outer layer 20 and 21 and different shapes.

In addition to footpads, the benefits of the essential oils and/or plant products may be obtained using single layer liners consisting of a single layer of absorbent natural or synthetic material onto which the oils are applied or into which the plant products are incorporated, and optionally, a leak-proof bottom covering. The liners may be removably attached to the inner sole of the footwear, for example by adhesive or Velcro, or may be permanently stitched into the footwear.

In another embodiment of the invention, the essential oils and/or plant products are formed within or on discrete inserts that are attached to the footwear at selected positions. The inserts may be permanently attached to the footwear, for example by stitching, or removably attached, using an adhesive or Velcro. The inserts may be formed in aesthetically pleasing shapes such as hearts, and may be placed in the footwear by the user at locations on the feet needing therapy, or at approximate locations on human feet that correspond to reflexology “points” of sensitivity.

As needed, the essential oils or plant products may be reapplied or reinserted into the footwear, either directly by spraying, or by replacing the materials in the insole footpads, liners or inserts, which can be constructed so as to be resealable, for example, by use of velcro or zipper closures.

As will be apparent to those skilled in the art in which the invention is addressed, the present invention may be embodied in forms other than those specifically disclosed without departing from the spirit or potential characteristics of the invention. Particular embodiments of the present invention described above are therefore to be considered in all respects an illustrative and not restrictive. The scope of the invention is as set forth in the appended claims and equivalents thereof, rather than being limited to the examples contained in the foregoing description.

We claim:

1. A method of providing essential oils and/or plant products to contact human feet comprising applying essential oils and/or plant products to footwear at selected locations corresponding to regions of the human feet.

2. The method of claim 1 wherein the essential oils and/or plant products are applied to components of footwear selected from the group consisting of insole footpads, liners and inserts.

3. The method of claim 2 wherein the components are removable.

4. The method of claim 2 wherein the essential oils and/or plant products are incorporated into a multilayered pad.

5. The method of claim 4 wherein the multilayered pad comprises an inner absorbent layer and an outer porous layer.

6. The method of claim 5 wherein the outer porous layer comprises synthetic or natural material.

7. The footwear made by the method of claim 1.

8. Footwear components made by the method of claim 2.

9. Footwear comprising essential oils and/or plant products applied to at least a portion of said footwear that contacts the user's feet.

10. The footwear of claim 9 wherein the essential oils and/or plant materials are applied to footwear components selected from the group consisting of insole pads, liners and inserts.

11. The footwear of claim 9 wherein the portions of said footwear that contact the user's feet correspond to reflexology points on human feet.
